(19) United States
(12) Patent Application Publication
(10) Pub. No.: US 2010/0099895 A1
(43) Pub. Date: Apr. 22, 2010

(54) PROCESS FOR ONE-STAGE PREPARATION OF 2-METHYLTETRAHYDROFURAN FROM FURFURAL OVER A CATALYST

(75) Inventors: Tobias Wabnitz, Mannheim (DE); Daniel Breuninger, Bobenheim-Roxheim (DE); Jens Heimann, Worms (DE); Rene Backes, Lampertheim (DE); Rolf Pinkos, Bad Duerkheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/521,033

(22) PCT Filed: Jun. 24, 2008
(86) PCT No.: PCT/EP2008/058039

§ 371 Date: Jun. 24, 2009

(30) Foreign Application Priority Data
Jul. 2, 2007 (EP) 07111507.5

(51) Int. Cl. C07D 307/08

(52) U.S. Cl. 549/429

(57) ABSTRACT

The present invention provides a process for preparing 2-methyltetrahydrofuran by one-stage hydrogenation of furfural with a hydrogen-comprising gas in the presence of a supported catalyst which comprises at least one noble metal from groups 8, 9 and/or 10 of the periodic table of the elements.

The present invention relates to a process for one-stage preparation of 2-methyltetrahydrofuran from furfural over a catalyst.

2-Methyltetrahydrofuran (referred to hereinafter as 2-Me-THF) is an organic solvent with high dissolution power. 2-Me-THF is used as a replacement solvent for chemical syntheses for tetrahydrofuran (referred to hereinafter as THF), from which it differs advantageously by its lower water solubility which decreases with increasing temperature, as a fuel additive since it is better miscible with common hydrocarbon-based fuels than alcoholic additives, and as a comonomer for the preparation of polyethers with improved properties over the homopolymers.

2-Me-THF is obtainable from renewable raw materials. 2-Me-THF can be obtained from plant wastes by digesting the hemicelluloses present to give furfural and converting it to 2-Me-THF, and thus contributes to sustainable development.

While the preparation of furfural from plant wastes, especially agricultural wastes, is known and has achieved a high level of development, the conversion of furfural to 2-Me-THF, in contrast, is yet to be solved in a technically satisfactory manner.

The elementary reactions of the hydrogenating conversion of furfural are known and have been described in detail by Zheng et al., Journal of Molecular Catalysis A: Chemical (2006), 246 (1-2), 18-23. The authors consider 2-methylfuran to be necessary as a precursor in the preparation of 2-Me-THF and describe main reactions and side reactions of the hydrogenation, including the formation of carbon monoxide by decarbonylation.

Even though 2-Me-THF frequently forms in small amounts in hydrogenating conversions of furfural, there are only few publications in which the direct conversion of furfural to 2-Me-THF is described.

Kyosuke et al., J. Pharm. Soc. Jpn 66 (1946), 58 show that the direct conversion of furfural to 2-methyl-THF over Raney nickel catalysts at 260° C. affords only small amounts of the product of value. Instead, Kyosuke et al. recommend as advantageous a two-stage process via methylfuran as an isolated intermediate and the use of various catalysts for the two stages. For instance, copper chromite according to Adkins is used in the first stage and Raney nickel in the second stage.

Proskuryakov et al., Trudy Leningradskogo Tekhnologicheskogo Instituta imeni Lensoveta (1958), 44, 3-5, describe a yield of not more than 42% 2-Me-THF in the conversion of furfural over a 1:1 mixture of a Raney nickel catalyst and a copper chromite catalyst in an autoclave at 220° C. and 160 atm. The authors show that higher 2-Me-THF contents cannot be obtained by this route owing to side reactions to give glycols and other ring-opening products of furfural not specified in detail.

Another disadvantage which has been recognized is the difficult isolation and purification of 2-Me-THF from the resulting reaction effluent mixtures, since the THF, 2-pentanone and water by-products, as pure substances or in the form of their azeotropes, have boiling points similar to that of 2-Me-THF. For instance, the boiling point of the water/2-Me-THF azeotrope is 73° C., that of the water/THF azeotrope is 64° C. and that of the water/2-pentanone azeotrope is 84° C., while the pure substance THF boils at 66° C., the pure substance 2-Me-THF boils at 80° C. and the pure substance 2-pentanone boils at 102° C.

U.S. Pat. No. 6,479,677 discloses a two-stage process for preparing 2-Me-THF using a separate catalyst in each case for each stage. Each stage is performed in a separate reactor with different catalysts. This gas phase process comprises the hydrogenation of furfural over a copper chromite catalyst to give methylfuran, which is then converted to 2-Me-THF over a nickel catalyst.

However, the process disclosed has a series of disadvantages. For instance, two catalysts and different reaction conditions are required for the individual stages of the reaction, which complicates the industrial performance and necessitates spatial separation of the individual reactors. The addition of hydrogen is required separately for each hydrogenation step, and the formation of carbon monoxide, which always proceeds in small amounts from furfural under thermal stress, leads to deactivation of the nickel catalyst and to the formation of highly toxic, volatile Ni(CO)₄. As a result of the accumulation of critical impurities such as carbon monoxide, the economically desirable cycle gas method becomes impossible.

It was therefore an object of the present invention to provide a process for one-stage preparation of 2-methyltetrahydrofuran from furfural using specific catalysts without isolation or purification of intermediates, with whose aid 2-methyltetrahydrofuran can be obtained especially by conversion in a reactor and in circulation mode in good yield and purity.

Accordingly, the present invention relates to a process for one-stage hydrogenation of furfural with a hydrogen-comprising gas in the presence of a supported catalyst which comprises at least one noble metal from groups 8, 9 and/or 10 of the periodic table, especially ruthenium, rhodium, iridium, gold, palladium and/or platinum, preferably palladium and/or platinum.

In this application, one-stage or one-stage hydrogenation is understood to mean a process which, proceeding from furfural, without isolation or purification of intermediates, leads to the 2-Me-THF end product.

In contrast to the prior art processes, the process according to the invention is advantageously carried out in one stage and over only one catalyst. The supported catalyst which comprises at least one noble metal from groups 8, 9 and/or 10 of the periodic table of the elements, preferably palladium and/or platinum, is the only catalyst used in the process according to the invention.

### Catalyst

The catalyst used in accordance with the invention has, as an active metal, at least one noble metal from groups 8, 9 and/or 10 of the periodic table of the elements, especially ruthenium, rhodium, iridium, gold, preferably palladium and/or platinum, more preferably palladium, on a support. The catalyst may additionally comprise metals from groups 4 and 7 to 12 of the periodic table of the elements and, if appropriate, elements of groups 1 and 2 of the periodic table of the elements, especially sodium, potassium, calcium or magnesium. It preferably does not have any further active metals apart from palladium and platinum.

The application of the active metals can be achieved by impregnating the support in aqueous metal salt solutions, for example aqueous palladium salt solutions, by spraying corresponding metal salt solutions onto the support or by other suitable processes, such as impregnation.

The catalytically active metals can be applied to the support material, for example, by impregnation with solutions or suspensions of the salts or oxides of the elements in question, drying and subsequent reduction of the metal compounds to give the metals or compounds of lower oxidation state in question by means of a reducing agent, preferably with hydrogen or complex hydrides. Another means of applying the catalytically active metals to these supports consists in impregnating the supports with solutions of thermally readily decomposable salts, for example with nitrates, or thermally readily decomposable complexes, for example carbonyl or hydride complexes of the catalytically active metals, and heating the impregnated supports thus obtained to temperatures of from 300 to 600° C. for the purpose of thermal decomposition of the adsorbed metal compounds. This thermal decomposition is preferably undertaken under a protective gas atmosphere. Suitable protective gases are, for example, nitrogen, carbon dioxide, hydrogen or the noble gases. In addition, the catalytically active metals can be deposited on the catalyst support by vapor deposition or by flame-spraying. The content in these supported catalysts of the catalytically active metals is in principle not critical for the success of the process according to the invention. However, higher contents of catalytically active metals generally lead to higher space-time yields than lower contents.

Suitable metal salts of platinum and palladium are the nitrates, nitrosylnitrates, halides, carbonates, carboxylates, acetylacetonates, chlorides, chloro complexes or amine complexes of the corresponding metals, preference being given to the nitrates.

In the case of catalysts which comprise palladium and platinum and possibly further active metals on the support, the metal salts or metal salt solutions can be applied simultaneously or successively.

The supports coated or impregnated with the metal salt solution are subsequently dried, preferably at temperatures between 100° C. and 150° C., and optionally calcined at temperatures between 200° C. and 600° C., preferably between 350° C. and 450° C. In the case of separate impregnation, the catalyst is dried after each impregnation step and optionally calcined as described above. The sequence in which the active components are applied by impregnation is freely selectable.

Subsequently, the coated and dried and optionally calcined supports are activated by treatment in a gas stream which comprises free hydrogen at temperatures between about 30° C. and about 600° C., preferably between about 150° C. and about 450° C. The gas stream preferably consists of from 50 to 100% by volume of H₂ and from 0 to 50% by volume of N₂.

The metal salt solution or solutions is/are applied to the support or supports in such an amount that the total content of active metal, based in each case of the total weight of the catalyst, is from about 0.1 to about 30% by weight, preferably from about 0.1 to about 10% by weight, more preferably from about 0.25 to about 5% by weight, and especially from about 0.5 to about 2.5% by weight.

Useable support metals include, for example, activated carbon, for example in the form of the commercial product Supersorbon carbon from Donau Carbon GmbH, 60388 Frankfurt am Main, aluminum oxide, silicon dioxide, silicon carbide, calcium oxide, titanium dioxide and/or zirconium dioxide or mixtures thereof, preference being given to using activated carbon.

### The process

The process according to the invention is notable in that the conversion is brought about in one stage and with only one catalyst.

The inventive one-stage hydrogenation can be performed in one or more, especially in two, three, four, five, six, seven, eight, reactors.

The reaction mixture preferably flows through the reactor or the reactors in each case from the top downward.

In the process according to the invention, the hydrogenation can be performed in the gas phase or the liquid phase; preference is given to working in the gas phase. In general, the process is performed in the gas phase at a temperature of from about 150 to 300° C., preferably from about 190 to 250° C. The pressures used are generally from 1 to 15 bar absolute, preferably from about 5 to 15 bar abs. The pressure in this application is reported as the total pressure or absolute (abs.) pressure.

In the liquid phase, the process according to the invention is performed generally at from 150 to 250° C. at pressures of from 20 to 200 bar abs.

The process according to the invention can be performed either continuously or batchwise, preference being given to the continuous performance of the process. In the continuous process, the amount of furfural provided for the hydrogenation is from about 0.05 to about 3 kg per liter of catalyst per hour, more preferably from about 0.1 to about 1 kg per liter of catalyst per hour.

The hydrogenation gases used may be any gases which comprise free hydrogen and do not comprise harmful amounts of catalyst poisons, for example CO. For example, it is possible to use reformer offgases. Preference is given to using pure hydrogen as the hydrogenation gas. However, it is also possible additionally to use inert carrier gases such as steam or nitrogen.

In the liquid phase, the inventive hydrogenation can be performed in the absence or presence of a solvent or diluent, i.e. it is not necessary to perform the hydrogenation in solution.

However, it is possible to use a solvent or diluent. The solvent or diluent used may be any suitable solvent or diluent. The selection is not critical provided that the solvent or diluent used is capable of forming a homogeneous solution with the furfural to be hydrogenated.

Examples of suitable solvents or diluents include the following: straight-chain or cyclic ethers, for example tetrahydrofuran or dioxane, and aliphatic alcohols in which the alkyl radical preferably has from 1 to 10 carbon atoms, especially from 3 to 6 carbon atoms.

The amount of the solvent or diluent used is not particularly restricted and may be selected freely as required, although preference is given to those amounts which lead to from 10 to 70% by weight solution of the furfural intended for the hydrogenation.

Furthermore, the hydrogenation reactor, in the case of performance of the hydrogenation in the liquid phase, can be operated in straight pass, i.e. without product recycling, or in circulation, i.e. a portion of the hydrogenation mixture leaving the reactor is conducted in a circuit.

In the case of performance of the inventive hydrogenation in the gas phase, the reaction products are condensed fully and removed after leaving the reactor. The gaseous fractions, hydrogen and any additional carrier gas used are returned partly to the reactor in circulation (cycle gas mode). In the preferred cycle gas mode, the ratio of cycle gas to fresh gas volumes is at least 1:1, preferably at least 5:1, more preferably at least 10:1.

Useful reactors include fixed bed reactors, for example tube bundle reactors. In the liquid method, it is possible to use fluidized bed reactors.

The reaction effluents of the inventive hydrogenation are condensed in a manner known per se, but preferably by cooling in a heat exchanger to from 0 to 80° C. After the condensation, a phase separation sets in. The lower phase consists of water to an extent of more than 90%, while the upper phase, as well as the desired 2-Me-THF product, comprises only small amounts of by-products which can be removed readily by any subsequent purifying distillation. 2-Methyltetrahydrofuran (2-Me-THF) is obtained by the process according to the invention in very good purity and yield. The phase separation can be effected at ambient temperature. However, the reaction effluents are preferably condensed at 60° C. since the miscibility of 2-methyl-THF and water is particularly low at this temperature.

The process according to the invention will now be illustrated in detail hereinafter with reference to a few working examples.

### EXAMPLES

### Catalyst Preparation Example

4 kg of Supersorbon carbon (4 mm extrudates, manufacturer: Donau Carbon GmbH) were initially charged in an impregnating drum and sprayed with 2.8 kg of a 7.2% by weight aqueous solution of palladium(II) nitrate, based on palladium, at room temperature with the aid of a fine nozzle (1 mm). The liquid was absorbed completely into the pores of the carbon support. The material was subsequently dried in a drying cabinet at 100° C. for 40 hours.

Subsequently, the dried catalyst was activated (reduced) in a water stream at 200° C.

The catalyst thus prepared comprised 5% by weight of palladium based on the weight of the catalyst.

### Analysis of the Hydrogenation Products

The 2-Me-THF, 2-pentanone, 3-pentanone, 1-pentanol, THF, furan and methylfuran reaction products and the furfural starting material were analyzed by gas chromatography. To this end, the mixtures were injected, diluted with methanol or acetone (dilution of from 1:10 to 1:100) or undiluted, into the GC chromatograph (from HP, carrier gas: hydrogen) onto a 30 m DB1 column (from J+W) and analyzed at oven temperatures of from 60° C. to 300° C. (heating rate 8 Kelvin per minute up to 220° C., then 20 Kelvin per minute up to 300° C.) with a flame ionization detector (temperature: 290° C.). The purity was determined by integrating the signals of the chromatogram.

### Example 1

In a system for continuous hydrogenation consisting of an evaporator, an oil-heated 3.8 l jacketed tubular reactor and a separator and a cycle gas compressor, furfural was hydrogenated continuously over fixed bed catalysts in the gas phase.

The tubular reactor was filled with 3 l (corresponding to 1350 g) of a Pd catalyst (5% Pd/Supersorbon, 4 mm extrudates).

The tubular reactor was flowed through from the top downward. The catalyst was activated with nitrogen/hydrogen mixtures at 200° C. at ambient pressure by the method known to those skilled in the art such that the content of hydrogen in the mixed gas was increased slowly from 0 up to 100%. Subsequently, the system was pressurized to 10 bar with hydrogen, fresh hydrogen gas was adjusted to 150 l (STP)/h, the evaporator was heated to 290° C., the reactor to 260° C., and the cycle gas was put into operation. 100 g/h of furfural which had been distilled in one stage were conveyed into the evaporator. During the hydrogenation, the cycle gas was adjusted to 1200 g/h, 95 l (STP)/h of offgas were sent to incineration. Under these conditions, over 260 h, furfural was converted to an extent of >99%; the selectivity for 2-MeTHF was 50%. The upper phase of the biphasic effluent had the following composition: furan 2.4% by weight, 2-methylfuran 2% by weight, THF 20% by weight, 2-MeTHF 49% by weight, 2-pentanone 9.2% by weight, 2-pentanol 0.5% by weight, 1-pentanol 0.9% by weight, remainder to 100% unidentified by-products. The by-products could be removed by means of distillation according to the prior art, such that the desired 2-MeTHF product was obtained in a purity of >99%.

### Example 2

In a system for continuous hydrogenation consisting of an evaporator, an oil-heated 0.375 l jacketed tubular reactor and a separator and a cycle gas compressor, furfural was hydrogenated continuously over fixed bed catalysts in the gas phase.

The tubular reactor was filled with 350 ml (corresponding to 173.3 g) of a Pd catalyst (5% Pd/Supersorbon, 4 mm extrudates).

The tubular reactor was flowed through from the bottom upward. The catalyst was activated with nitrogen/hydrogen mixtures at 260° C. by the method known to those skilled in the art such that the content of hydrogen in the mixed gas was increased slowly from 0 up to 100%. Subsequently, the system was pressurized to 10 bar with hydrogen, fresh hydrogen gas was adjusted to 150 l (STP)/h, and the evaporator was heated to 240° C., the reactor to 245° C. During the hydrogenation, 20 g/h of furfural were conducted over the catalyst from the bottom upward (liquid phase mode). 35 l (STP)/h of fresh gas and 550 l (STP)/h of cycle gas were used. Under these conditions, furfural was converted completely.

The reaction effluents were admixed with tetraethylene glycol dimethyl ether in a manner known per se by metering 25 ml/h of tetraethylene glycol dimethyl ether into the gas stream between tubular reactor and separator. After decompression of the stream in the ambient pressure part, the liquid phase which comprised the reaction products was removed and collected in the separator.

Neglecting the tetraethylene glycol dimethyl ether, the monophasic reaction effluent contained: 2-methylfuran 59% by weight, THF 31% by weight, 2-MeTHF 49% by weight, 2-pentanol 0.5% by weight, n-butanol 0.5% by weight, remainder to 100% unidentified by-products. The by-products could be removed by means of distillation, such that the desired 2-MeTHF product was obtained in a purity of >99%.

1-9. (canceled)

10. A process for preparing 2-methyltetrahydrofuran by one-stage hydrogenation of furfural with a hydrogen-comprising gas in the presence of a supported catalyst which comprises, as an active metal, at least one noble metal selected from ruthenium, rhodium, iridium, gold, palladium and platinum, and optionally additionally comprises metals from groups 1, 2 and 4 of the periodic table of elements.

11. The process according to claim 10, wherein the catalyst does not comprise any further active metals apart from palladium and platinum.

12. The process according to claim 10, wherein the catalyst comprises palladium and/or platinum and at least one element from groups 1 and 2 of the periodic table of the elements.

13. The process according to claim 10, wherein activated carbon, aluminum oxide, silicon dioxide, silicon carbide, calcium oxide, titanium dioxide and/or zirconium dioxide or mixtures thereof are used as the support.

14. The process according to claim 10, which is performed in the liquid phase at a temperature of from 150 to 250° C. at from 20 to 200 bar absolute.

15. The process according to claim 14, which is performed in the presence or absence of a solvent.

16. The process according to claim 10, which is performed in the gas phase at a temperature of from 150 to 300° C. at from 1 to 15 bar absolute.

17. The process according to claim 10, which is performed in cycle gas mode or circulation mode.
